(19) United States
(12) Patent Application Publication
(10) Pub. No.: US 2011/0112148 A1
(43) Pub. Date: May 12, 2011

(54) INDOLINE COMPOUNDS

(75) Inventors: Jose Falco, Barcelona (ES); Albert Palomer, Barcelona (ES); Antonio Guglietta, Molins De Rei (ES)

(73) Assignee: Ferrer Internacional S.A.

(21) Appl. No.: 12/739,666

(22) PCT Filed: Oct. 23, 2008
(86) PCT No.: PCT/EP2008/064389

§ 371 Date: Dec. 30, 2010

(30) Foreign Application Priority Data
Oct. 25, 2007 (ES) P200702798

(51) Int. Cl. A61K 31/4439; A61K 31/405; C07D 401/02; C07D 209/04; A61P 25/06; A61P 25/00; A61P 25/20; A61P 25/28; A61P 25/16; A61P 25/18; A61P 3/04; A61P 3/10; A61P 25/08

(52) U.S. Cl. 514/339; 514/415; 546/277.4; 548/490

(57) ABSTRACT

This invention provides new 2,3-dihydro-indole compounds, their use for the treatment or prevention of melatonin-ergic disorders and its compositions.

### FIELD OF THE ART

The present invention belongs to the field of compounds with activity on melatonin receptors, specifically indolins (2,3-dihydro-1H-indoles), and more specifically acylated 6-(alkoxy or phenylalkoxy)-2,3-dihydro-indol-1-yl-alkylamines.

### STATE OF THE ART

Insomnia is the most common sleep disorder and affects 20-40% of adults, with a frequency that increases with age. Insomnia has many causes. One of these is the interruption of the normal wakefulness-sleep cycle. This dyssynchrony may result in pathological changes. A potential therapeutic treatment that allows correcting said effect consists in re-synchronising the wakefulness-sleep cycle by modulating the melatoninergic system (Li-Qiang Sun, Bioorganic & Medicinal Chemistry Letters 2005, 15, 1345-49).

Melatonin is a hormone segregated by the pineal gland that is responsible for information on the light-dark cycles, for controlling the circadian rhythm in mammals and for modulating retinal physiology. Melatonin synthesis and its nightly secretion are controlled by the suprachiasmatic nucleus and synchronised by environmental light (Osamu Uchikawa et al., J. Med. Chem. 2002, 45, 4222-39; Pandi-Perumal et al., Nature Clinical Practice 2007, 3 (4), 221-228).

Melatonin secretion in humans occurs simultaneously to sleep at night, and the increase in melatonin levels is correlated with the increase in the desire to sleep during the evening.

In humans, the clinical applications of melatonin range from treatment of the delayed sleep phase syndrome to jet lag treatment, including treatment applied to night shift workers and as a hypnotic treatment.

Melatonin receptors have been classified as MT1, MT2 and MT3 based on pharmacological profiles. The MT1 receptor is located in the hypothalamus central nervous system, whereas the MT2 receptor is distributed throughout the central nervous system and the retina. The presence of MT1 and MT2 receptors has been described at the peripheral level. The MT1 and MT2 receptors are involved in a large amount of pathologies, the most representative of these being depression, stress, sleep disorders, anxiety, seasonal affective disorders, cardiovascular pathologies, digestive system pathologies, insomnia or fatigue due to jet lag, schizophrenia, panic attacks, melancholia, appetite disorders, obesity, insomnia, psychotic diseases, epilepsy, diabetes, Parkinson's disease, senile dementia, disorders associated to normal or pathological aging, migraine, memory loss, Alzheimer's disease and brain circulation disorders. The MT3 receptor has been recently characterised as the homologue of the quinone reductase-2 (QR2) enzyme. MT1 and MT2 are G protein-coupled receptors (GPCR), the stimulation of which by an agonist leads to a reduction in adenylate cyclase activity and the resulting reduction in intracellular cAMP.

U.S. Pat. No. 4,600,723 and U.S. Pat. No. 4,665,086 advocate the use of melatonin to minimise alterations of the circadian rhythms that occur due to changes in work shifts from days to nights or from passing quickly through several time zones in an airplane (jet lag). Several families of compounds with melatoninergic activity had been described in patent documents EP 848699B1, U.S. Pat. No. 5,276,051, U.S. Pat. No. 5,308,866, U.S. Pat. No. 5,708,005, U.S. Pat. No. 6,034,239 (ramelteon), U.S. Pat. No. 6,143,789, U.S. Pat. No. 6,310,074, U.S. Pat. No. 6,583,319, U.S. Pat. No. 6,737,431, U.S. Pat. No. 6,908,931, U.S. Pat. No. 7,235,550, WO 8901472 and WO 2005062992.

U.S. Pat. No. 5,633,276 describes compounds for the treatment of melatoninergic system alterations belonging to formula:

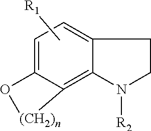

where the substituents R₁ and R₂ and the variable n have the meanings described therein, the preferred compound being that of example 7 (R₁═H, R₂═(CH₂)₂—NHCOCH₃, n=2).

Ramelteon, N42-[(8S)-1,6,7,8-tetrahydro-2H-indeno[5,4-b]furan-8-yl)ethyl]propionamide, is the first melatonin agonist introduced in therapy. It is indicated in insomnia and its mechanism of action is based on the agonism of the MT1 and MT2 receptors.

Ramelteon is a non-selective compound against MT1 and MT2, and selective against other receptors at the central and peripheral level. Its Ki is 0.014 nM for MT1 and 0.045 nM for MT2. It shows good absorption, but experiences an important first-pass metabolic effect. It is biotransformed into four metabolites, one of these being M-II, active and with an important distribution volume. Ramelteon clearance is 88%.

The research of new melatonin agonists that may be useful in the treatment of insomnia responds to a fundamental health need, and therefore justifies continued research for compounds with improved properties.

Therefore, the present invention is aimed at new acylated 6-(alkoxy or phenylalkoxy)-2,3-dihydro-indol-1-yl-alkylamines that are active against melatonin receptors, especially MT1 and MT2 receptors. As a result, the compounds of the present invention are useful in the treatment and prevention of all those diseases that are mediated by MT1 and MT2 receptors. Some non-limiting examples of melatoninergic disorders are depression, stress, sleep disorders, anxiety, seasonal affective disorders, cardiovascular pathologies, digestive system pathologies, insomnia or fatigue due to jet lag, schizophrenia, panic attacks, melancholia, appetite disorders, obesity, insomnia, psychotic diseases, epilepsy, diabetes, Parkinson's disease, senile dementia, disorders associated to normal or pathological aging, migraine, memory loss, Alzheimer's disease and brain circulation disorders.

### DETAILED DESCRIPTION OF THE INVENTION

The present invention relates to indoline compounds of general formula I:

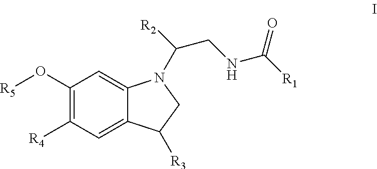

wherein:
R₁ is a radical chosen from the group consisting in a linear or branched (C₁-C₆) alkyl, (C₃-C₆) cycloalkyl and CF₃;
R₂ is hydrogen or a linear or branched (C₁-C₆) alkyl radical;
R₃ is hydrogen or a linear or branched (C₁-C₆) alkyl radical;
R₄ is a radical chosen from the group consisting of hydrogen, a halogen atom, phenyl and pyridyl;
R₅ is a radical chosen from the group consisting of linear or branched alkyl(C₁-C₆) and (CH₂)ₙ-Ph; and
n is an integer from 1 to 6; and pharmaceutically acceptable salts and hydrates thereof.

Pharmaceutically acceptable salts are those that may be administered to a patient, such as a mammal (e.g. salts with acceptable safety in mammals for a given dosing regimen). Such salts may be obtained from pharmaceutically acceptable inorganic and organic bases and from pharmaceutically acceptable inorganic and organic acids. The salts obtained from pharmaceutically acceptable inorganic bases include ammonium, calcium, copper, ferric and ferrous salts, lithium, magnesium, manganic and manganous salts, potassium, sodium, zinc salts and the like. Especially preferred are the ammonium, calcium, magnesium, potassium and sodium salts. The salts obtained from pharmaceutically acceptable organic bases include primary, secondary and tertiary amine salts, including substituted amines, cyclic amines, natural amines and the like, such as arginine, betaine, caffeine, choline, N,N′-dibenzylethylendiamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. The salts obtained from pharmaceutically acceptable acids include acetic, ascorbic, benzene sulphonic, benzoic, camphorsulphonic, citric, ethanesulphonic, edisylic, fumaric, gentisic, gluconic, glucuronic, glutamic, hippuric, hydrobromic, hydrochloric, isethionic, lactic, lactobionic, maleic, malic, mandelic, methanesulphonic, mucic, naphthalenesulphonic, naphthalene-1,5-disulphonic, naphthalene-2,6-disulphonic, nicotinic, nitric, orotic, pamoic, pantothenic, phosphoric, succinic, sulphuric, tartaric, p-toluenesulphonic, xinafoic and the like. Particularly preferred are citric, hydrobromic, hydrochloric, isethionic, maleic, naphthalene-1,5-disulphonic, phosphoric, sulphuric and tartaric acids.

The specific compounds of Formula I are chosen from the group consisting of:
1) N-[2-(6-methoxy-2,3-dihydro-indol-1-yl)-ethyl]-acetamide;
2) N-[2-(6-methoxy-2,3-dihydro-indol-1-yl)-ethyl]-propionamide;
3) [2-(6-methoxy-2,3-dihydro-indol-1-yl)-ethyl]-cyclopropanecarboxamide;
4) 2,2,2-trifluoro-N-[2-(6-methoxy-2,3-dihydro-indol-1-yl)-ethyl]-acetamide;
5) N-[2-(6-methoxy-2,3-dihydro-indol-1-yl)-propyl]-acetamide;
6) N-[2-(6-methoxy-3-methyl-2,3-dihydro-indol-1-yl)-ethyl]-acetamide;
7) N-[2-(5-bromo-6-methoxy-2,3-dihydro-indol-1-yl)-ethyl]-acetamide;
8) N-[2-(6-methoxy-5-pyridin-4-yl-2,3-dihydro-indol-1-yl)-ethyl]-acetamide;
9) N-[2-(6-methoxy-5-phenyl-2,3-dihydro-indol-1-yl)-ethyl]-acetamide;
10) N-[2-(6-phenethyloxy-2,3-dihydro-indol-1-yl)ethyl]-acetamide;
11) [2-(6-phenethyloxy-2,3-dihydro-indol-1-yl)-ethyl]-cyclopropanecarboxamide;
12) N-[2-(6-phenethyloxy-2,3-dihydro-indol-1-yl)-ethyl]-propionamide;
13) N-{2-[6-(3-phenyl-propoxy)-2,3-dihydro-indol-1-yl]-ethyl}-acetamide;
14) N-{2-[6-(3-phenyl-propoxy)-2,3-dihydro-indol-1-yl]-ethyl}-butyramide;
15) N-{2-[6-(3-phenyl-propoxy)-2,3-dihydro-indol-1-yl]-ethyl}-propionamide;
16) {2-[6-(3-phenyl-propoxy)-2,3-dihydro-indol-1-yl]-ethyl}-cyclopropanecarboxamide;
17) 2,2,2-trifluoro-N-{2-[6-(3-phenyl-propoxy)-2,3-dihydro-indol-1-yl]-ethyl}-acetamide; and
18) N-{2-[6-(4-phenyl-butoxy)-2,3-dihydro-indol-1-yl]-ethyl}-acetamide.

Table 1 shows the meaning of the substituents for each compound:

TABLE 1

| Example | R₁ | R₂ | R₃ | R₄ | R₅ |
|---|---|---|---|---|---|
| 1 | Me | H | H | H | Me |
| 2 | Et | H | H | H | Me |
| 3 | cPr | H | H | H | Me |
| 4 | CF₃ | H | H | H | Me |
| 5 | Me | Me | H | H | Me |
| 6 | Me | H | Me | H | Me |
| 7 | Me | H | H | Br | Me |
| 8 | Me | H | H | 4-pyridyl | Me |
| 9 | Me | H | H | pH | Me |
| 10 | Me | H | H | H | Ph—(CH₂)₂ |
| 11 | cPr | H | H | H | Ph—(CH₂)₂ |
| 12 | Et | H | H | H | Ph—(CH₂)₂ |
| 13 | Me | H | H | H | Ph—(CH₂)₃ |
| 14 | Pr | H | H | H | Ph—(CH₂)₃ |
| 15 | Et | H | H | H | Ph—(CH₂)₃ |
| 16 | cPr | H | H | H | Ph—(CH₂)₃ |
| 17 | CF₃ | H | H | H | Ph—(CH₂)₃ |
| 18 | Me | H | H | H | Ph—(CH₂)₄ |

Another aspect of the present invention is to provide the use of a specific compound from Table 1 to prepare a medicinal product for the treatment or prevention of melatoninergic disorders. Said melatoninergic disorders are chosen from depression, stress, sleep disorders, anxiety, seasonal affective disorders, cardiovascular pathologies, digestive system pathologies, insomnia or fatigue due to jet lag, schizophrenia, panic attacks, melancholia, appetite disorders, obesity, insomnia, psychotic diseases, epilepsy, diabetes, Parkinson's disease, senile dementia, disorders associated to normal or pathological aging, migraine, memory loss, Alzheimer's disease and brain circulation disorders.

Another aspect of the present invention is to provide pharmaceutical compositions comprising a specific compound from Table 1 and one or more pharmaceutically acceptable excipients.

Another aspect of the present invention is to provide the use of said pharmaceutical compositions in the preparation of a medicinal product for the treatment or prevention of melatoninergic disorders. Said melatoninergic disorders are chosen from depression, stress, sleep disorders, anxiety, seasonal affective disorders, cardiovascular pathologies, digestive system pathologies, insomnia or fatigue due to jet lag, schizophrenia, panic attacks, melancholia, appetite disorders, obesity, insomnia, psychotic diseases, epilepsy, diabetes, Parkinson's disease, senile dementia, disorders associated to normal or pathological aging, migraine, memory loss, Alzheimer's disease and brain circulation disorders.

How to obtain compounds of general formula I is described in the following diagrams, wherein the substituents R₁, R₂, R₃, R₄, R₅ and R₆ are as described above.

Diagram 1 describes the synthetic strategy corresponding to the introduction of substituent R₁, shown for R₂═R₃═R₄═H and R₅=Me.

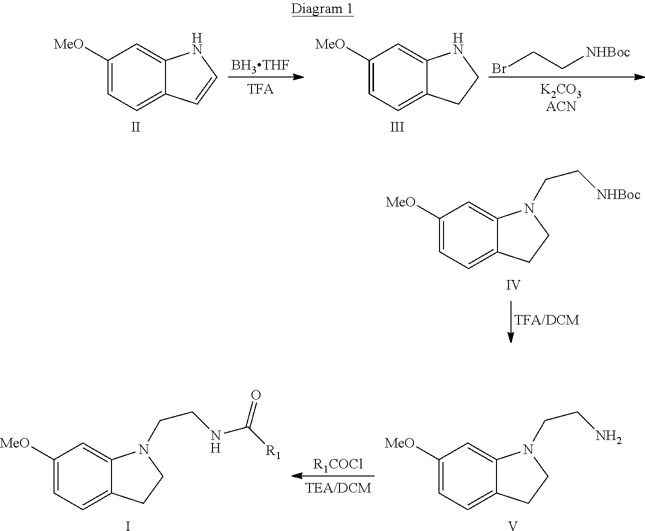

First, indoline III is obtained from commercially available indol II by the use of borane in tetrahydrofurane (THF). Said indoline is alkylated with 2-bromoethylamine protected with Boc in potassium carbonate in acetonitrile (ACN). Having obtained the protected compounds IV, the corresponding intermediate amines V are obtained by reaction with trifluoroacetic acid (TFA) in dichloromethane (DCM). Finally, the last step consists in a usual coupling between the amines V and acid chlorides to yield compounds I.

The use of substituted bromoacetonitriles is necessary for the introduction of R₂ substituents in the side chain. Diagram 2 shows the corresponding synthesis pathway, shown for R₃═R₄═H and R₅=Me.

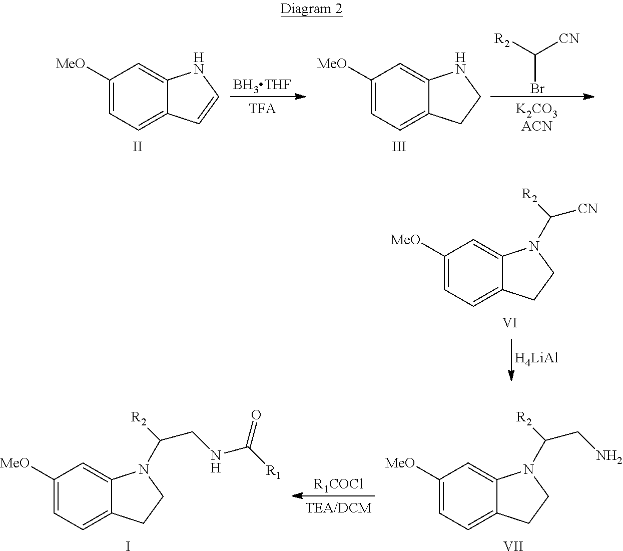

The difference with Diagram 1 above lies in the alkylation step. In this case the alkylating agent is a substituted bromoacetonitrile. In the case where R₂ is methyl, said derivative is commercially available. Amines VII can be produced after obtaining VI by reduction with lithium and aluminium hydride and aluminium. Said amines follow the same coupling procedure as that described in Diagram 1.

When R₃ is different than hydrogen, it is necessary to follow the synthetic pathway described in Diagram 3. This pathway describes the particular case when R₂═R₄═H and R₃═R₅=Me.

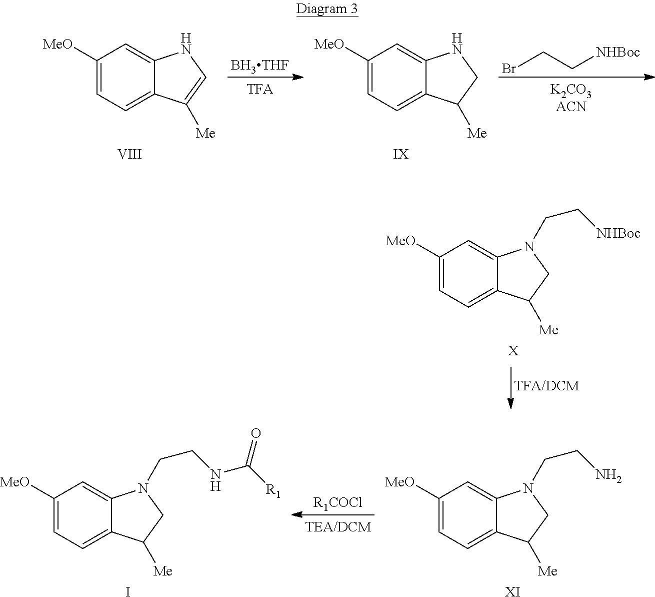

The starting indol VIII is commercially available. For R₃ groups that are different from methyl it is probable that the corresponding indoles are also commercially available. Otherwise, a selective alkylation at position 3 of indol II may be performed with the corresponding halogenated derivative, using a strong base such as sodium hydride.

The introduction of R₄ substituents other than hydrogen is detailed in Diagram 4, shown for R₁═R₅=Me and R₂═R₃═H.

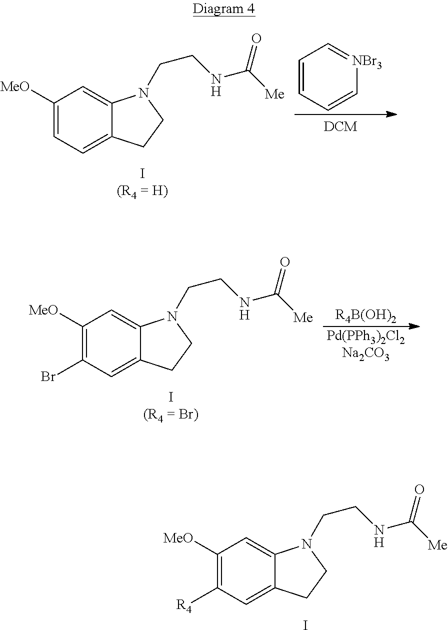

As can be observed, the compounds selectively brominated at position 5 of the indoline ring are obtained by reaction of starting indoline I (R₄ hydrogen) with pyridinium perbromide in dichloromethane. Said brominated derivatives, by Suzuki reaction using the corresponding boronic acids, allow obtaining indolines I substituted at position 5.

Finally, Diagram 5 shows the synthetic pathway to produce O-substituted indolines I at R₅, shown for R₂═R₃═R₄═H.

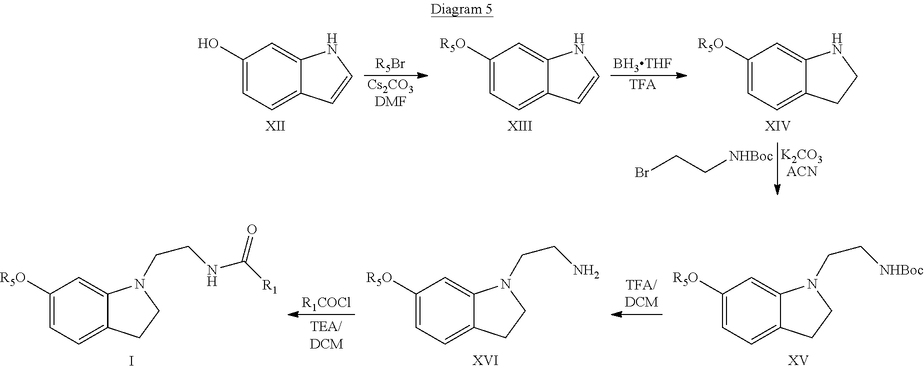

The only difference with the synthetic procedures described above in diagrams 1-3 lies in the first step. We must start from 6-hydroxyindole XII, which by selective Williamson alkylation at the oxygen atom produces alkoxyindoles XIII. Having obtained the alkoxyindoles XIII, indolines I can be obtained following the chemistry described above, i.e. reduction to indoline, N-alkylation to introduce the side chain, deprotection and subsequent coupling with acid chlorides.

Pharmaceutical compositions comprising compounds of the present invention include those that are adequate for oral, rectal and parenteral administration (including the subcutaneous, intramuscular and intravenous routes), although the most suitable route will depend on the nature and seriousness of the pathology being treated. The preferred administration route for the compounds of the present invention is frequently the oral route.

The active ingredients can be mixed with one or more pharmaceutical excipients following conventional pharmaceutical techniques for formulation. Several excipients can be used according to the pharmaceutical form to be prepared. Liquid oral compositions (such as, for example, suspensions, solutions, emulsions, aerosols and mouthwashes) may use, for example, water, glycols, oils, alcohols, flavour enhancers, preservatives, colorants and the like. Solid oral compositions use, for example, starches, sugars (such as, for example, lactose, sucrose and sorbitol) celluloses (such as, for example, hydroxypropyl cellulose, carboxymethyl cellulose, ethyl cellulose and microcrystalline cellulose), talc, stearic acid, magnesium stearate, dicalcium phosphate, rubbers, copovidone, surfactants such as sorbitan monooleate and polyethyleneglycol, metallic oxides (such as, for example, titanium dioxide and ferric oxide) and other pharmaceutical diluents such as water. Homogeneous preformulations are thus formed containing the compounds of the present invention.

In the case of the preformulations the compositions are homogeneous, such that the active ingredient is dispersed uniformly in the composition, which can therefore be divided in equal unit doses such as tablets, coated tablets, powders and capsules.

Tablets and capsules are most advantageous oral forms due to their ease of administration. Tablets can be coated using aqueous or nonaqueous conventional techniques if so desired. A large variety of materials can be used to form the coating. Such materials include a large number of polymeric acids and their mixtures with other components such as, for example, shellac, cetyl alcohol and cellulose acetate.

Liquid forms in which the compounds of the present invention can be incorporated for oral or injectable administration include aqueous solutions, capsules filled with fluid or gel, syrups with flavour enhancers, aqueous suspensions in oil and emulsions flavoured with edible oils such as, for example, cottonseed oil, sesame oil, coconut oil or peanut oil, as well as mouthwashes and similar pharmaceutical carriers. Suitable dispersing or suspension agents for the preparation of aqueous suspensions include synthetic and natural gums such as tragacanth, Acacia, alginates, dextranes, sodium carboxymethylcellulose, methylcellulose, polyethyleneglycol, polyvinylpyrrodidone or gelatin.

A suitable dosage range to be used is a total daily dose from 0.1 to 500 mg approximately, more preferably from 1 mg to 100 mg, either in a single administration or in separate doses if necessary.

### EMBODIMENTS OF THE INVENTION

The present invention is additionally illustrated by means of the following examples, which do not intent to limit the scope thereof.

### Example of Pharmacological Assessment 1

### Determination of the Agonist Activity on MT1 Receptors

In order to screen compounds for the MT1 receptor a cell line is used that is characterised by stable overexpression of the recombinant human MT1 receptor in a cell line that in turn co-expresses mitochondrial apoaequorin and the Gα16 subunit.

The Ga16 subunit belongs to the G protein family, formed by GPCR, wherein the transduction of intracellular signals occurs via phospholipase (PLC). PLC activation produces an increase in inositol-triphosphate levels that leads to an increase in intracellular calcium. Ga16 overexpression thus allows an increase in intracellular calcium levels that is independent and compatible with the study receptor's own signal transduction pathway.

Apoaequorin is the inactive form of aequorin, a phosphoprotein that requires a hydrophobic prosthetic group, coelenterazine, to produce the active form. Following its binding to calcium, the aequorin oxidises coelenterazine to coelenteramide, a reaction that releases CO₂ and light.

The trial protocol for the screening of possible agonists consists in collecting the cells and keeping them in suspension overnight in the presence of coelenterazine in order to reconstitute aequorin. On the following day the cells are injected on a plate where the compounds to be screened are diluted, and the luminescence released is read immediately. When wishing to study the possible antagonism of the same compounds, the reference agonist compound is added in the same well after 15-30 min from the first injection and the luminescence released is assessed.

Agonist activity is calculated as percentage activity with respect to the reference agonist at the concentration corresponding to its EC100. Antagonist activity is expressed as percentage inhibition over the reference agonist activity at the concentration corresponding to its EC80.

### Example of Pharmacological Assessment 2

### Determination of Agonist Activity on MT2 Receptors

In order to study agonism against MT2 receptors we use a recombinant cell line that expresses these receptors and coexpresses mitochondrial apoaequorin and the Ga16 subunit, as in the model used for MT1 screening. The compounds of the present invention show in this model that they also have agonism for the MT2 receptors.

Table 2 shows the results for agonism on MT1 receptors versus the standard N-[2-(2,3,7,8-tetrahydro-1H-furo[2,3-g]indol-1-yl)-ethyl]-acetamide (U.S. Pat. No. 5,633,276, example 7).

TABLE 2

| | MT1 | |
|---|---|---|
| Compound | 100 nm | 1 nm |
| Example 1 | 92.0 | 13.1 |
| Example 4 | 94.0 | 14.6 |
| Example 5 | 89.8 | 11.5 |
| N-[2-(2,3,7,8-tetrahydro-1H-furo[2,3-g]indol-1-yl)- | 76.6 | 13.9 |
| ethyl]-acetamide (U.S. Pat. No. 5,633,276, example 7) | | |

In short, the present invention provides new compounds that, despite having certain structural similarity with compounds of the state of the art, surprisingly show greater agonist activity on the MT1 receptor, which implies superior therapeutic properties.

### Reference Example 1

### General Procedure for Obtaining Indolines III

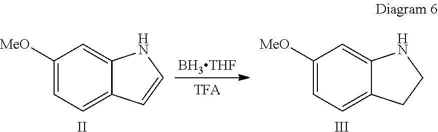

3 g (20 mmol) of 6-methoxyindole II are dissolved at 0° C. in 30 mL of borane solution in 1M THF (30 mmol). It is purged with nitrogen atmosphere and stirred for 30 min at 0° C. 30 mL of TFA are added and it is stirred for 30 min at 0° C. Once the stirring is finished the reaction is finished by adding 6M NaOH until it reaches a basic pH. The crude product is extracted with DCM. 2.90 g (Yield=100%) of the indoline III are obtained as a yellowish oil.

HPLC-MS: Purity 99.9%, M+1=150

### Reference Example 2

### General Procedure for Obtaining Indolines IV

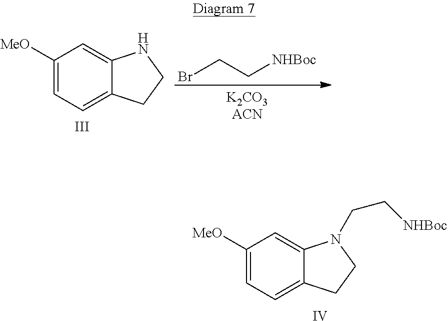

0.67 g (4.99 mmol) of the indoline III are dissolved in 15 mL of acetonitrile. 2.01 g (8.98 mmol) of the bromoderivative and 1.86 g (13.47 mmol) of potassium carbonate are added. It is heated at 80° C. for 12 h. It is allowed to cool and the solvent is eliminated under low pressure. 50 mL of water and 50 mL of DCM are added and the organic phase is extracted. The organic phase is dried over anhydrous magnesium sulphate and filtered. It is evaporated and 629 mg (Yield=43%) of indoline IV are obtained as a yellowish oil.

HPLC-MS: Purity 99.9%, M+1=293

### Reference Example 3

### General Procedure for Obtaining Deprotected Indolines V

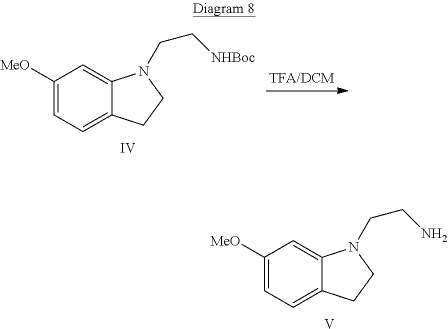

0.25 g (0.85 mmol) of the indoline IV are dissolved in 5 mL of DCM. 0.69 mL (8.5 mmol) of TFA are added. It is stirred at room temperature for 2 h. The solvent is eliminated under low pressure. The residue thus obtained is suspended in DCM and washed with a saturated solution of sodium carbonate. The organic phase is dried over anhydrous magnesium sulphate and filtered. It is evaporated and 160 mg (Yield=100%) of the amine V are obtained as a yellowish oil.

HPLC-MS: Purity 99.9%, M+1=193

### Reference Example 4

### General Procedure for Obtaining Indolines I

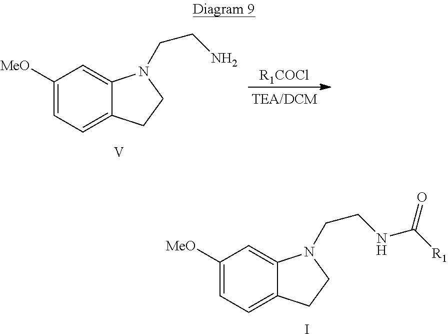

160 mg of amine V (0.85 mmol) are dissolved in 20 mL of anhydrous DCM. 0.339 mL of triethylamine (TEA) (2.436 mmol) are slowly added and subsequently 0.93 mmol of the corresponding acid chloride are also slowly added. Stir at room temperature for 2 h and 30 min. 5 mL of 1N HCl area added and it is stirred for 10 min. Separate the organic phase and dry. It is evaporated to dryness and the corresponding amides I are obtained.

Example for R₁═CF₃: 220 mg (Yield=90%) are obtained

HPLC-MS: Purity 94%, M+1=289

The compounds thus obtained are detailed in the following Table 3.

TABLE 3

| Example | R₁ | R₂ | R₃ | R₄ | R₅ | LCMS Purity (%) | M + 1 |
|---|---|---|---|---|---|---|---|
| 1 | Me | H | H | H | Me | 96 | 235 |
| 2 | Et | H | H | H | Me | 92 | 249 |
| 3 | cPr | H | H | H | Me | 100 | 261 |
| 4 | CF₃ | H | H | H | Me | 94 | 289 |

### Reference Example 5

### General Procedure for Obtaining Indolines VI

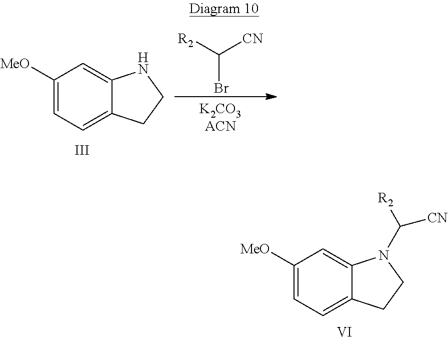

0.51 g (3.4 mmol) of the indoline III are dissolved in 10 mL of acetonitrile. 0.59 mL (16.8 mmol) of the bromoderivative and 1.41 g (10 mmol) of potassium carbonate are added. It is heated at 80° C. for 12 h. It is allowed to cool and the solvent is eliminated under low pressure. 50 mL of water and 50 mL of DCM are added and the organic phase is extracted. The organic phase is dried over anhydrous magnesium sulphate and filtered. The residue thus obtained is purified by column chromatography using hexane/ethyl acetate as an eluant. 0.27 mg (Yield=39%) of indoline VI are obtained as a yellowish oil.

HPLC-MS: Purity 99.9%, M+1=203

### Reference Example 6

### General Procedure for Obtaining Indolines VII

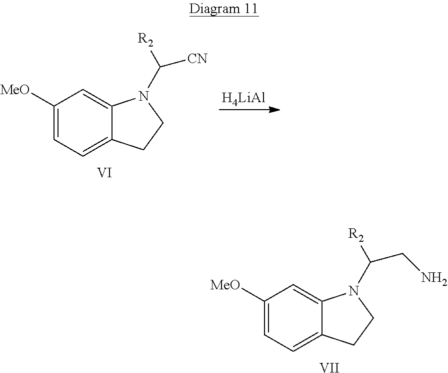

76 mg (2 mmol) of the lithium and aluminium hydride are dissolved in 5 mL of anhydrous THF under a nitrogen atmosphere and in an ice bath. A solution of 0.27 g (1.33 mmol) of the indoline VI is added dropwise into 5 mL of THF. It is stirred at 0° C. for 1 h, removed from the ice bath, and stirred again for 1 h at room temperature. Water and 1N NaOH are added until reaching a basic pH. The alumina formed over Celite® is filtered. The filtrate is extracted with DCM. The organic phase is dried over anhydrous magnesium sulphate and filtered. 0.21 mg (Yield=78%) of the indoline VII are obtained as a yellowish oil.

HPLC-MS: Purity 99.9%, M+1=207

The last step in the synthesis corresponds to the coupling with acid chloride, described above. We therefore provide an example of a compound of this subfamily corresponding to the specific case in which R₂ is methyl. The details are shown in Table 4.

TABLE 4

| Example | R₁ | R₂ | R₃ | R₄ | R₅ | LCMS Purity (%) | M + 1 |
|---|---|---|---|---|---|---|---|
| 5 | Me | Me | H | H | Me | 94 | 249 |

The procedure is the same when R₃ is other than hydrogen (Table 5).

TABLE 5

| Example | R₁ | R₂ | R₃ | R₄ | R₅ | LCMS Purity (%) | M + 1 |
|---|---|---|---|---|---|---|---|
| 6 | Me | H | Me | H | Me | 95 | 249 |

### Reference Example 7

### General Procedure for Obtaining Brominated Indolines I

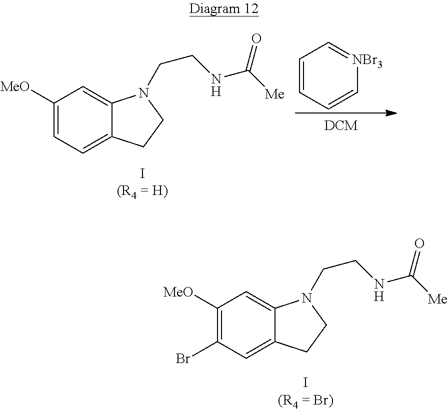

70 mg (0.30 mmol) of the starting compound I are dissolved in 10 mL of DCM and 96 mg (0.30 mmol) of pyridinium perbromide are added. It is stirred at room temperature for 1 h. The reaction crude is evaporated and it is purified by flash chromatography using DCM/MeOH as an eluant. 80 mg (Yield=85%) of a yellow oil identified as I (R₅=Br) are obtained.

HPLC-MS: Purity 96%, M+1=314

### Reference Example 8

### General Procedure for Obtaining Compounds I

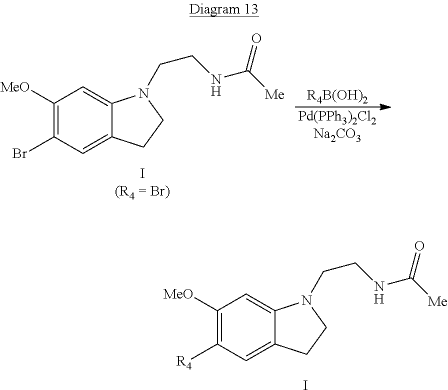

0.15 g (0.48 mmol) of the brominated amide I are dissolved in 20 mL of dimethoxyethane and it is purged with an inert argon atmosphere. The tip of a spatula of palladium-dichloro-bis(triphenylphosphine) is added and also 0.86 mmol of the corresponding boronic acid and 0.43 mL of a solution of 0.86 mmol of sodium carbonate in 1 mL of water. Stir at 75° C. for 3 h. Allow to cool and add 100 mL of water. Extract with 50 mL of DCM. Dry, filter and evaporate the organic phase. The residue thus obtained is purified by reverse-phase preparative chromatography, using acetonitrile/water as an eluant. The type I products in the form of a yellowish oil are thus obtained.

The compounds thus obtained are detailed in the following Table 6.

TABLE 6

| | | | | | | LCMS | |
|---|---|---|---|---|---|---|---|
| Example | R₁ | R₂ | R₃ | R₄ | R₅ | Purity (%) | M + 1 |
| 7 | Me | H | H | Br | Me | 96 | 314 |
| 8 | Me | H | H | 4- | Me | 92 | 312 |
| | | | | pyridyl | | | |
| 9 | Me | H | H | pH | Me | 100 | 311 |

### Reference Example 9

### General Procedure for Obtaining O-Alkylated Indolines XIII

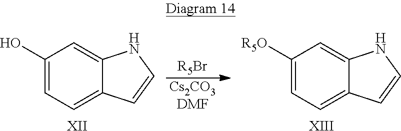

The 6-hydroxyindole XII (2.85 g, 21 mmol) is dissolved in 50 mL of DMF. 7.67 g (23 mmol) of caesium carbonate and 23 mmol of the corresponding halogenated derivative are added. It is heated at 80° C. for 2 h. Allow to cool and filter the reaction crude. Evaporate to dryness under low pressure and dissolve in DCM. Wash with 1N NaOH. Separate the organic phase, filter and evaporate. The XIII derivatives are thus obtained in solid form.

Example when R₆=PhCH₂CH₂CH₂: 3.10 g are obtained (Yield: 59%).

HPLC-MS: Purity 99.9%, M+1=251

The type XIII compounds follow the reactions described in Diagram 1 from this point.

The compounds thus obtained are detailed in the following Table 7.

TABLE 7

| Example | R₁ | R₂ | R₃ | R₄ | R₅ | LCMS Purity (%) | M + 1 |
|---|---|---|---|---|---|---|---|
| 10 | Me | H | H | H | Ph-(CH₂)₂ | 325 | 93 |
| 11 | cPr | H | H | H | Ph-(CH₂)₂ | 351 | 100 |
| 12 | Et | H | H | H | Ph-(CH₂)₂ | 339 | 100 |
| 13 | Me | H | H | H | Ph-(CH₂)₃ | 339 | 95 |
| 14 | Pr | H | H | H | Ph-(CH₂)₃ | 368 | 91 |
| 15 | Et | H | H | H | Ph-(CH₂)₃ | 353 | 92 |
| 16 | cPr | H | H | H | Ph-(CH₂)₃ | 365 | 91 |
| 17 | CF₃ | H | H | H | Ph-(CH₂)₃ | 393 | 98 |
| 18 | Me | H | H | H | Ph-(CH₂)₄ | 353 | 98 |

1. Indoline compounds chosen from the group consisting of:
1) N-[2-(6-methoxy-2,3-dihydro-indol-1-yl)-ethyl]-acetamide;
2) N-[2-(6-methoxy-2,3-dihydro-indol-1-yl)-ethyl]-propionamide;
3) [2-(6-methoxy-2,3-dihydro-indol-1-yl)-ethyl]-cyclopropanecarboxamide;
4) 2,2,2-trifluoro-N-[2-(6-methoxy-2,3-dihydro-indol-1-yl)-ethyl]-acetamide;
5) N-[2-(6-methoxy-2,3-dihydro-indol-1-yl)-propyl]-acetamide;
6) N-[2-(6-methoxy-3-methyl-2,3-dihydro-indol-1-yl)-ethyl]-acetamide;
7) N-[2-(5-bromo-6-methoxy-2,3-dihydro-indol-1-yl)-ethyl]-acetamide;
8) N-[2-(6-methoxy-5-pyridin-4-yl-2,3-dihydro-indol-1-yl)-ethyl]-acetamide;
9) N-[2-(6-methoxy-5-phenyl-2,3-dihydro-indol-1-yl)-ethyl]-acetamide;
10) N-[2-(6-phenethyloxy-2,3-dihydro-indol-1-yl)-ethyl]-acetamide;
11) [2-(6-phenethyloxy-2,3-dihydro-indol-1-yl)-ethyl]-cyclopropanecarboxamide;
12) N-[2-(6-phenethyloxy-2,3-dihydro-indol-1-yl)-ethyl]-propionamide;
13) N-{2-[6-(3-phenyl-propoxy)-2,3-dihydro-indol-1-yl]-ethyl}-acetamide;
14) N-{2-[6-(3-phenyl-propoxy)-2,3-dihydro-indol-1-yl]-ethyl}-butyramide;
15) N-{2-[6-(3-phenyl-propoxy)-2,3-dihydro-indol-1-yl]-ethyl}-propionamide;
16) {2-[6-(3-phenyl-propoxy)-2,3-dihydro-indol-1-yl]-ethyl}-cyclopropanecarboxamide;
17) 2,2,2-trifluoro-N-{2-[6-(3-phenyl-propoxy)-2,3-dihydro-indol-1-yl]-ethyl}-acetamide; and
18) N-{2-[6-(4-phenyl-butoxy)-2,3-dihydro-indol-1-yl]-ethyl}-acetamide;
and pharmaceutically acceptable salts and hydrates thereof.

2. The use of a compound of claim 1 to prepare a medicinal product for the treatment or prevention of melatoninergic disorders.

3. The use of claim 2 wherein said melatoninergic disorders are chosen from depression, stress, sleep disorders, anxiety, seasonal affective disorders, cardiovascular pathologies, digestive system pathologies, insomnia or fatigue due to jet lag, schizophrenia, panic attacks, melancholia, appetite disorders, obesity, insomnia, psychotic diseases, epilepsy, diabetes, Parkinson's disease, senile dementia, disorders associated to normal or pathological aging, migraine, memory loss, Alzheimer's disease and brain circulation disorders.

4. A pharmaceutical composition comprising a compound of claim 1 and one or more pharmaceutically acceptable excipients.

5. The use of the pharmaceutical composition of claim 4 to prepare a medicinal product for the treatment or prevention of melatoninergic disorders.

6. The use of claim 5 wherein said melatoninergic disorders are chosen from depression, stress, sleep disorders, anxiety, seasonal affective disorders, cardiovascular pathologies, digestive system pathologies, insomnia or fatigue due to jet lag, schizophrenia, panic attacks, melancholia, appetite disorders, obesity, insomnia, psychotic diseases, epilepsy, diabetes, Parkinson's disease, senile dementia, disorders associated to normal or pathological aging, migraine, memory loss, Alzheimer's disease and brain circulation disorders.

7. A method of treating or preventing melatoninergic disorders which comprises administering an effective amount of one or more compounds of claim 1 to a patient.
